(19) United States
(12) Patent Application Publication
(10) Pub. No.: US 2022/0087600 A1
(43) Pub. Date: Mar. 24, 2022

(54) SYSTEM AND METHOD FOR MEASURING AND MANAGING SLEEP EFFICACY IN A NON-INVASIVE MANNER

(71) Applicant: The Regents of the University of California, Oakland, CA (US)

(72) Inventors: Ian Campbell, Kensington, CA (US); Irwin Feinberg, Kensington, CA (US)

(73) Assignee: The Regents of the University of California, Oakland, CA (US)

(21) Appl. No.: 17/417,892

(22) PCT Filed: Dec. 30, 2019
(86) PCT No.: PCT/US19/68976

§ 371 Date: Jun. 24, 2021

Related U.S. Application Data
(60) Provisional application No. 62/785,810, filed on Dec. 28, 2018.

(51) Int. Cl. A61B 5/00; A61B 5/16; A61B 5/374

(52) U.S. Cl. CPC A61B 5/4815; A61B 5/163; A61B 5/4812; A61B 5/7221; A61B 5/374

(57) ABSTRACT

Systems and techniques are described herein for determining sleep adequacy in a non-invasive manner. In accordance with one embodiment of the present invention, this may be done by determining waking electroencephalogram (EEG) power density of a user. Described herein is a sleep quality measuring device configured to be worn by a user that includes EEG sensors configured to contact an occipital aspect of the user's head to determine an EEG power density of the user to determine sleep adequacy.

### CROSS-REFERENCE TO RELATED APPLICATIONS

This application claims priority o U.S. Provisional Patent Application No. 62/785,810 (Attorney Docket No. UCDAP009P), filed Dec. 28, 2018 and titled “SYSTEM AND METHOD FOR MEASURING AND MANAGING SLEEP EFFICACY IN A NON-INVASIVE MANNER,” which is incorporated herein by this reference for all purposes.

### TECHNICAL FIELD

The present disclosure relates generally to methods and systems for measuring and managing sleep using non-invasive means.

### BACKGROUND

Despite sleep's recognized biological importance, it has been difficult to demonstrate changes in brain physiology with reduced sleep durations. It would be desirable to have a non-invasive test for detecting where a subject has had biologically sufficient sleep.

### BRIEF DESCRIPTION OF THE DRAWINGS

The disclosure may best he understood by reference to the following description taken in conjunction with the accompanying drawings, which illustrate various examples.

FIGS. 1-3 are graphs showing electroencephalogram (EEG) measurements, in accordance with some embodiments.

FIG. 4A illustrates an example of a system for determining sleep adequacy, in accordance with some embodiments.

FIG. 4B illustrates an example of an electroencephalogram array, in accordance with some embodiments.

FIG. 5 illustrates an example of a sleep quality measuring device, in accordance with some embodiments.

FIGS. 6A and 6B illustrates examples of a user wearing a sleep quality measuring device, in accordance with some embodiments.

FIG. 7 is a flowchart illustrating a technique of using the sleep quality measurements, in accordance with some embodiments.

FIG. 8 illustrates a block diagram of a computer system capable of implementing various processes described herein, in accordance with some embodiments.

### DESCRIPTION OF PARTICULAR EMBODIMENTS

Described herein are systems and techniques for determining sleep adequacy in a non-invasive manner. In accordance with one embodiment of the present invention, this may be done by determining waking electroencephalogram (EEG) power density of a user. Thus, for example, a sleep quality measuring device may interface with a user (e.g., by being worn by the user) and determine a waking EEG power density of the user to determine sleep adequacy.

In a study of adolescents, sleep durations were reduced by restricting time in bed for four nights of either 10, 8.5 or 7 hours. Sleep reduction significantly decreased waking EEG power in a wide range of frequencies with both eyes closed and eyes open in central and occipital leads. As such, the reduced EEG power may result from insufficient metabolic recovery with insufficient sleep duration. In one embodiment, non-invasive tests utilize the fact that graded reductions in sleep durations produce graded reductions in waking EEG power in human subjects to determine the sleep adequacy of the user.

Reference will now be made in detail to some specific examples of the present disclosure. While the present disclosure is described in conjunction with these specific embodiments, it will be understood that it is not intended to limit the present disclosure to the described embodiments. On the contrary, it is intended to cover alternatives, modifications, and equivalents as may he included within the spirit and scope of the present disclosure as defined by the appended claims.

In the following description, numerous specific details are set forth in order to provide a thorough understanding of the present disclosure. Particular example embodiments of the present disclosure may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. Furthermore, the techniques and mechanisms of the present disclosure will sometimes describe a connection between two entities. It should be noted that a connection between two entities does not necessarily mean a direct, unimpeded connection, as a variety of other entities may reside between the two entities. Consequently, a connection does not necessarily mean a direct, unimpeded connection unless otherwise noted.

The biological function of sleep is that it is a period of reduced neural activity that permits restoration of metabolic substrates required for waking brain function. Although rapid eye movement (REM) sleep is characterized by normal or even elevated neural activity, direct measurements of glucose uptake in humans show that in non-REM sleep, which makes up 75-80% of adult human sleep, brain metabolism declines below waking levels by more than 30%. This reduced metabolic activity is consistent with the hypothesis that one function of sleep is to allow replenishment of needed energy substrates. As a result, it is expected to find that sleep restriction impairs brain energetics during subsequent waking.

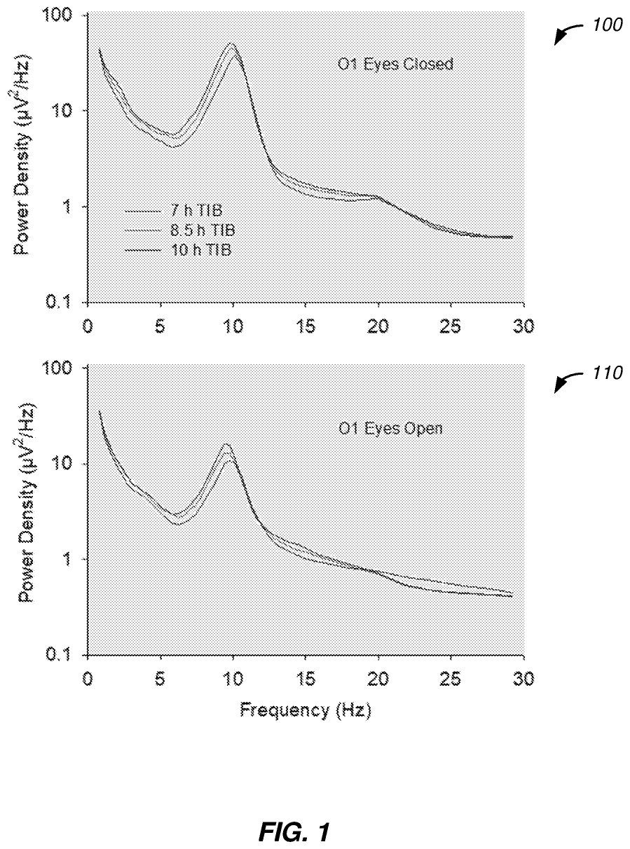

Evidence for this has been found in longitudinal studies with sleep restrictions in adolescents. Such evidence is described in FIGS. 1-3. FIGS. 1-3 are graphs showing electroencephalogram (EEG) measurements, in accordance with some embodiments. One aspect of this evidence is that sleep restriction dose-dependently reduced waking EEG power across a wide range of frequencies. These reductions were significant and detectable with both eyes opened as well as eyes closed in occipital and central EEG leads.

The experimental protocol included recordings of waking EEG from subjects (Ss) during a day of laboratory testing which followed 4 consecutive nights with 10, 8.5 or 7 hours of time in bed (TIB). At the first of 3 annual recordings (the study took place over three years), the initial cohort of 77 Ss (36 female, 41 male) ranged in age from 9.9 to 14.0 y (mean=12.2 y). Attrition reduced the cohort to 76 Ss in year 2 and to 67 in year 3.

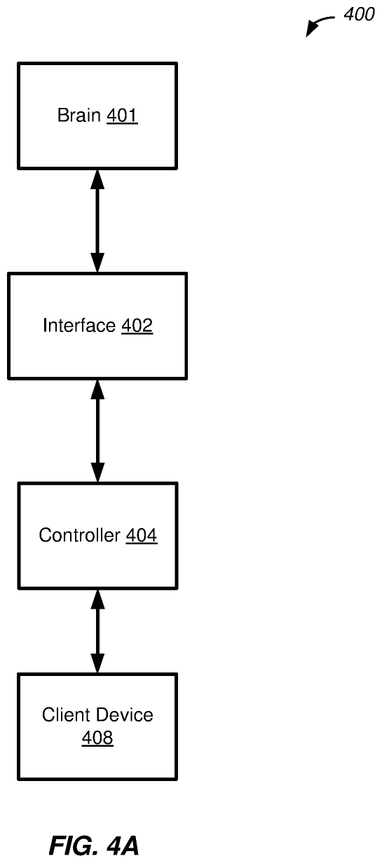

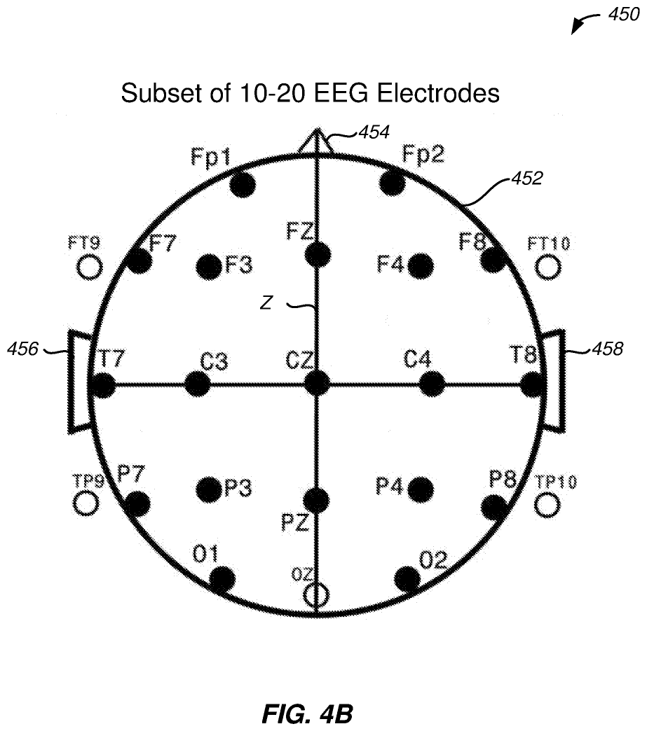

Annually, each S completed each of the three 4-night TIB protocols. Continuous sleep EEG recordings on the second and fourth nights of each TIB condition documented that controlling TIB produced the expected effects on sleep durations; these averaged (+/−se) 530+/−2, 471+/−2 and 405+/−1 min for 10, 8.5 and 7 h TIB, respectively. Starting at 0900 on the day following the fourth experimental night, various tests of daytime sleepiness and cognitive performance were administered four times at 2 hour intervals. One test measured alpha EEG attenuation, which previous investigators found is diminished by sleepiness (7, 8). This test was performed by recording waking EEG for 3 minutes with eyes open while Ss stared at a dot on the wall. Ss then closed their eyes for 2 minutes, opened their eyes and stared at the dot for another 2 minutes, and finally closed their eyes for an additional 2 minutes. Monopolar EEG from occipital (O1, O2), parietal (P3, P4), central (C3, C4), and frontal (F3, F4) leads were recorded, as such in FIG. 4B. These waking EEG tests were performed four times at 2 h intervals beginning at 9 AM. Based on prior alpha attenuation and Karolinska Drowsiness Test reports, the analyses focused on the occipital and central EEG. The results presented below are based on all 4 recordings for each TIB condition for each year. The results are statistically evaluated with mixed effects analyses that included all completed recordings.

FIG. 1 below shows the EEG power spectra for the three TIB conditions for the O1 lead with eyes open and eyes closed. EEG power density (PD) in most frequencies in O1 and C3 was highest after 10 h in bed and lowest after 7 hours, with the 8.5 TIB condition intermediate. PD was greater with eyes closed (as shown in plot 100) than with eyes open (as shown in plot 110) in central as well as occipital leads.

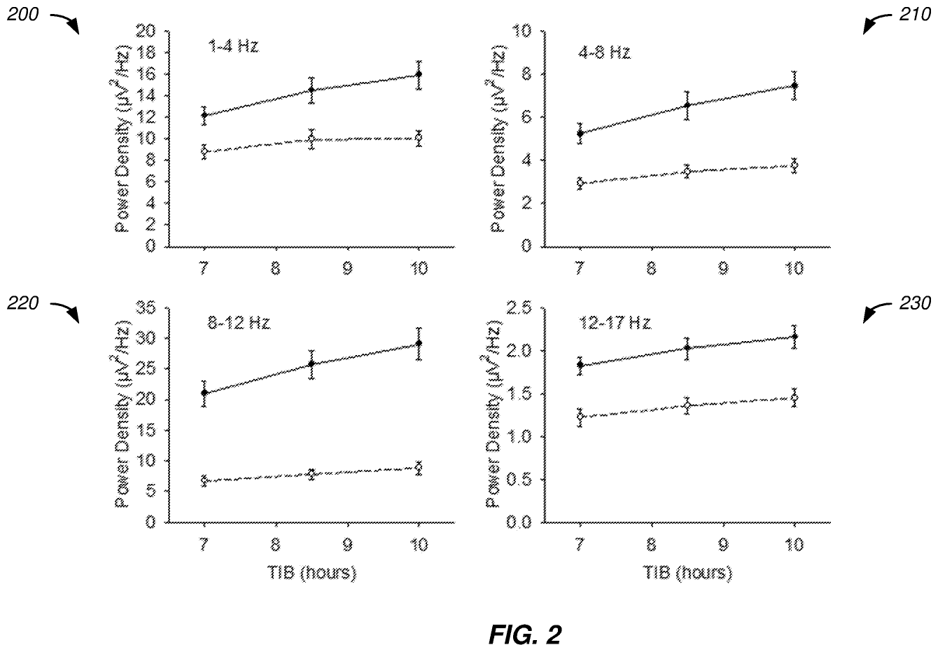

FIG. 2 plots PD in O1 for the main frequency bands in the waking EEG for eyes open and eyes closed, and Table 1 summarizes the tests of statistical significance. (Results for right-sided scalp leads (O2 and C4) are virtually identical to those of the left side.)

In O1, PD was reduced by TIB restriction for all frequency bands from delta through low beta with both eyes open and eyes closed; PD was significantly higher with eyes closed. (Traditional Greek-letter nomenclature is used for these groups of frequencies.) High beta (17-30 Hz) PD in O1 was not significantly affected by sleep restriction although it did show a strongly significant increase with eye closure. As indicated by the significant TIB by eyes interaction and as shown in FIG. 2, the TIB effect on delta, theta, and alpha EEG power was stronger in the eyes closed condition.

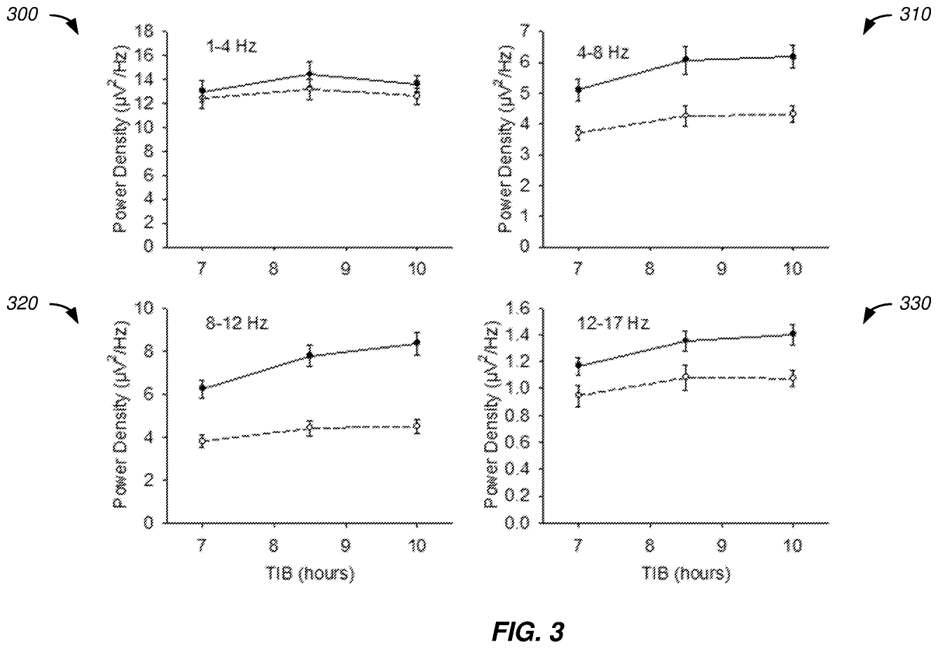

Delta power density in C3, in contrast to PD in O1, was not significantly reduced by TIB although theta through low beta in C3 was reduced (as shown in FIG. 3). Another finding presented in Table 1, a highly significant maturational decrease in PD with age, is consistent with prior cross-sectional and longitudinal age-studies of waking EEG. It is also consistent with our longitudinal study of sleep EEG (13) which further documented the long-known massive decline in non-REM delta power across this age range.

One of the findings of the study is that eye closure increases EEG power in many waking frequencies. This is in addition to its well-known effect on alpha, and that this response occurs in leads anterior to the occipital, is consistent with the observations of previous studies. Both groups found that eye closure increases waking EEG power in multiple areas over a wide range of frequencies. In one study addressing direct brain recordings with subdural electrodes is especially compelling. They found that eye closure increased PD in subgamma frequencies in occipital, parietal, temporal and frontal cortices and both hippocampi, as well as producing focal decreases in high frequency (gamma) power in the occipital lobe. Thus, suppression of EEG PD with opening the eyes may be due to widespread activation of information processing systems, rather than as simple changes in global arousal levels.

The longitudinal study described herein provides data that demonstrate a dose dependent response of human brain electrophysiology to graded durations of sleep. The reduction in brain wave power when sleep is restricted is consistent with the view that one function of sleep is to provide metabolic support for waking brain functions.

On a practical level, these changes in power density with varied sleep durations enable a biological, non-invasive test for sleep sufficiency, such as through the systems and techniques described herein. Such tests allow for a variety of treatment techniques and diagnostic applications. Such treatment techniques and applications may include, for example, research on sleep and cognition, aging, hypnotic efficacy, whether adolescents are getting adequate sleep at different ages, whether certain sleep treatment techniques are effective, whether an insomniac is receiving enough sleep, and/or whether military and public service personnel have obtained the amount of sleep needed to perform critical tasks. The systems and techniques described herein allow for measurement of EEG power in short sessions and rapid analysis of the EEG power to determine sleep adequacy. Based on the determination of whether the sleep received by the user is adequate or not, changes to treatment plans may be determined.

In one embodiment, a sleep quality measuring device is described. The sleep quality measuring device may include a body, an EEG sensor, and a controller. The body may be configured to couple to a head of a user and may include a body occipital portion configured to be disposed next to an occipital aspect of the head of the user. The EEG sensor may be disposed on the body occipital portion and configured to contact the occipital aspect when the sleep quality measuring device is worn by the user to detect EEG readings from the occipital aspect of the head of the user and output corresponding EEG data. The controller may be configured to perform operations including receiving the EEG data from the EEG sensor, determining, from the EEG data, a current alpha power of the user, comparing the current alpha power to a reference alpha power, determining a sleep adequacy rating based on the comparing, and outputting the sleep adequacy rating.

In another embodiment, a method of treatment is described. The method may include receiving, within a threshold period of time (e.g., between 30 minutes and two hours) after a user has awoken when the user is free of sleep associated drugs, EEG data from a sleep quality measuring device placed on a head of the user, where the sleep quality measuring device is placed to dispose an EEG sensor of the sleep quality measuring device to contact an occipital aspect of the head of the user, determining, from the EEG data, a current alpha power of the user, comparing the current alpha power to a reference alpha power, determining a sleep adequacy rating based on the comparing, and providing an adjustment to a sleep treatment schedule based on the sleep adequacy rating.

In a further embodiment, a method of detecting sleep adequacy is described. The method comprises placing a plurality of contact leads that detect EEG data on a subject's occipital aspect of the head bilaterally after the subject has woken up for a period of time (e.g., 10 to 30 minutes) and has their eyes closed; transmitting the EEG data to a processing device; analyzing the EEG data for sleep adequacy by comparing the absolute power of the alpha wave in the 8-12 Hz range and assessing whether there is a decrease in alpha power compared to the subject's reference value after adequate sleep; and alerting the subject whether they have had adequate sleep to perform a function.

Methods of evaluating sleep adequacy using an EEG machine to measure the power of theta (4-8 Hz) and alpha (8-12 Hz) waves upon waking (waking EEG) are also described. In some embodiments, the methods are based on detecting a phenomenon that occurs when an individual wakes up and wherein the alpha wave activity (8-12 Hz) increases when the eyes are closed (hereinafter referred to as “Waking Eyes Closed Alpha”). Waking Eyes Closed Alpha can he measured by Alpha Power (μV2) on waking EEG. In some embodiments, a physical device to determine whether an individual has had adequate sleep for a number of purposes ranging from assessing whether it is safe for an individual to perform certain operations (e.g., operating equipment) or evaluating the efficacy of a sleep drug is described.

In one embodiment, administering a waking EEG on an individual after an adequate night of sleep in order to determine a reference Waking Eyes Closed Alpha value, measured as Alpha Power (μV2), is described. This reference value may be measured by placing EEG electrodes bilaterally on the individual's occipital region of the head upon waking up.

For subsequent days, the same individual can use the device to measure the Alpha Power of Waking Eyes Closed Alpha to determine whether the individual has had adequate sleep. In the described embodiment, this may need to be self-administered by the individual since the Waking Eyes Closed Alpha measurement needs to be made within a threshold period of time (e.g., between 30 minutes to an hour after the individual has awoken) after the individual wakes up.

The device may execute an algorithm to compare the current Alpha Power of Waking Eyes Closed Alpha to the individual's reference value to determine whether the individual has had adequate sleep. Based on the comparison of the current Alpha Power to the reference value, a determination may be made as to whether the individual has had sufficient sleep (e.g., whether the current Alpha Power is within a threshold percentage, such as within 70%, 90%, 100%, 120%, or some other value, of the reference). The device may interpret the results and provide a determination, such as “sufficient sleep,” “insufficient sleep,” “requires X hours more sleep,” etc., depending on device application.

In the waking EEG, alpha frequency power increases when eyes are closed, and this increase is diminished following sleep deprivation. One current longitudinal study varies time in bed (TIB) to determine changes in sleep need across adolescence. The effects of sleep restriction and age on waking alpha power in early adolescence are reported below.

In one embodiment, seventy seven children, age 9.85 to 14.0 years (mean=12.2, sd=1.2) at the time of first recording, were studied in the first year of the ongoing longitudinal study noted above. A laboratory day of performance and sleepiness testing follows four nights with TIB restricted to 7, 8.5 or 10 hours. Each participant completed all three sleep schedules. Laboratory days entailed 4 test sessions, every 2 hours starting at 0900. Each test session includes recording of waking EEG: 3 minutes with eyes open, followed by 2 minutes eyes closed, followed by 2 minutes eyes open, followed by 2 minutes eyes closed. EEG recorded from O1 and O2 was analyzed with FFT on 5 second artifact free epochs.

The results from this longitudinal study are described below. Following the 7 h TIB schedule, O1 alpha power with eyes open (57 μV2) increased (by 114 μV2) when eyes were closed (p<0.0001). This eyes closed effect increased by 12 μV2 for each additional hour of nighttime TIB (p=0.0002). There was no age effect on eyes open alpha, on the eyes closed effect, or on the TIB x eyes closed interaction (p>0.4 for all). Results for O2 EEG were similar.

The test may conclude that in young adolescents, sleep restriction diminishes the eyes closed increase in waking alpha EEG power. This finding raises the possibility that alpha power is a sensitive indicator of sleep recovery. The absence of an age effect contrasts starkly with MSLT findings from the same subjects where sleep extension provided a much stronger decrease in sleep likelihood in younger subjects.

The longitudinal study shows experimental evidence that waking EEG power is an indicator of sleep adequacy. The graphs below show that prior sleep restriction greatly reduces power across a wide range of frequencies in the waking EEG. Some embodiments may require modification of methods of ambulatory EEG recording and FFT (spectral) analysis of the output of an EEG recorder to determine power density in the main EEG frequency bands. EEG recording requires the application of surface electrodes to the scalp. The systems described herein include a device that applies surface electrodes to certain areas of the scalp.

There is evidence showing that all except a few EEG frequencies show reduced spectral power when sleep duration is reduced below an optimal amount. Some embodiments of this invention may require that waking EEG be recorded for several minutes in the morning after a night of adequate sleep duration (which would vary with age). This EEG is then analyzed by the sleep quality measuring device. The resulting power spectrum provides the standard, for that subject to evaluate other time in bed durations under consideration. In one embodiment, the data may be supplemented with recording data in small numbers of adults and, possibly, elderly subjects.

Referring back to FIG. 1, FIG. 1 shows a waking EEG power spectra for O1 with eyes closed and eyes open on the day following 4 consecutive nights of 3 different TIB schedules. Increasing TIB produced an overall increase in power density (F1,76=83.4, p<0.0001). The TIB effect differed by frequency band (F44,2.2×105=33.3, p<0.0001).

Also, with respect to FIG. 1, occipital (O1 or O2) waking EEG mean power density (n=77 subjects, up to 12 recordings per subject per time in bed condition) is plotted against frequency for eyes closed and eyes open conditions. EEG power density was reduced when prior time in bed was decreased from 10 (blue line) to 8.5 (green) or 7 (red) hours. The reduction is strongest in the eyes closed condition for EEG frequencies between 2 and 10 Hz and is greater for the 7 hr than for the 8.5 hr sleep restriction. Note that power density is plotted on a log scale (see FIG. 2 for the % effect).

Now referring back to FIG. 2, FIG. 2 shows the effect of time in bed (TIB) on mean (+/−se) O1 waking EEG power density in four frequency bands for both the eyes closed (solid line, filled circles) and eyes opened (dashed line, open circles) conditions.

Also, with respect to FIG. 2, to better demonstrate the reduction in waking EEG with prior sleep restriction, the power following 10 h in bed is expressed as a percent of the power following 7 h in bed. Power was significantly higher in all frequencies between 2 and 10 Hz in the 10 h time in bed condition. For both O1 and O2, eyes closed EEG power in the 9-10 Hz band after 10 h in bed was, on average, twice that after 7 h in bed. This is the largest non-invasively measured brain change that has been measured with sleep restriction in human subjects.

Now referring back to FIG. 3, FIG. 3 shows the effect of time in bed (TIB) duration on mean (+/−se) C3 waking EEG power density in four frequency bands for both the eyes closed (solid line, filled circles) and eyes opened (dashed line, open circles) conditions.

The tables below detail mixed effect analysis of time in bed (TIB), age, and eyes closed effects on power of waking EEG recorded from O1 and C3. Significance level is bold for positive effects (e.g. increasing power with increasing TIB), italicized for negative effects (e.g. decreasing power with increasing age), and plain text for non-significant (α=0.01) effects.

O1

| | | | Eyes | TIB * |
|---|---|---|---|---|
| Band | TIB | Age | closed | Eyes |
| Delta 1-4 H7 | <0.0001 | <0.0001 | <0.0001 | <0.0001 |
| Theta 4-8 Hz | <0.0001 | <0.0001 | <0.0001 | 0.0010 |
| Alpha 8-12 Hz | <0.0001 | <0.0001 | <0.0001 | <0.0001 |
| Beta 12-17 Hz | <0.0001 | <0.0001 | <0.0001 | 0.35 |
| Beta 17-30 H7 | 0.031 | <0.0001 | <0.0001 | 0.35 |

C3

| | | | Eyes | TIB * |
|---|---|---|---|---|
| Band | TIB | Age | closed | Eyes |
| Delta 1-4 Hz | 0.022 | <0.0001 | <0.0001 | 0.054 |
| Theta 4-8 Hz | <0.0001 | <0.0001 | <0.0001 | 0.052 |
| Alpha 8-12 Hz | <0.0001 | <0.0001 | <0.0001 | <0.0001 |
| Beta 12-17 Hz | <0.0001 | <0.0001 | <0.0001 | 0.25 |
| Beta 17-30 Hz | 0.013 | <0.0001 | <0.0001 | 0.23 |

FIG. 4A illustrates an example of a system for determining sleep adequacy, in accordance with some embodiments. In various embodiments, system 400 may be implemented for providing closed loop or open loop control in analysis of sleep adequacy and related treatments. In some embodiments, system 400 includes an interface, such as interface 402. In various embodiments, interface 402 is a brain interface that is configured to be coupled with a brain of a user, such as brain 401. As described herein, such coupling may include a sleep quality measuring device coupled to the head of the user and configured to sense EEG power of the user. In some embodiments, interface 402 includes one or more electrodes, as may be included in an electrode array. In other examples, interface 402 may include various sensors such as eye sensors to determine eye movement of the user. In various embodiments, such measured signals may be electrical signals derived based on neural activity of the brain of the user.

FIG. 4B illustrates an example of an electroencephalogram array, in accordance with some embodiments. FIG. 4B illustrates an example of standard electroencephalogram (EEG) electrode array 450 which may be implemented with various embodiments. FIG. 4B depicts standard EEG electrode names and positions along the head 452 of a user in vertex view with nose 454 above, left ear 456 to left, and right ear 458 to right. In various embodiments, EEG array 450, or a portion thereof, may be implemented as interface 402.

In various embodiments, EEG array 450 includes any number of electrodes. Such electrodes may include midline electrodes on midline Z, including FZ, Midline Frontal; CZ, Midline Central; PZ, Midline Parietal; OZ, Midline Occipital. In various embodiments, even numbers refer to right hemisphere locations, and odd numbers refer to left hemisphere locations including: Fp, Frontopolar (Fp4 and Fp2); F, Frontal (F3, F4, F7, F8); C, Central (C3, C4); T, Temporal (T7, T8); P, Parietal (P3, P4, P7, P8); O, Occipital (O4, O2). The standard 49, 40 to 20 electrodes are shown as black points. An additional subset of five, 40-40 electrodes are shown as open circles including FT, Frontotemporal (FT9, FT40); TP, Temporoparietal (TP9, TP40), and OZ. In various embodiments, EEG array 450 may include fewer or additional electrodes than shown in FIG. 4B, such as only sensors within one or more occipital positions (e.g., O1 and O2).

Referring back to FIG. 4A, in some embodiments, interface 402 further includes one or more dedicated processors and an associated memory configured to obtain and store the measurements acquired by portions of interface 402, such as the EEG sensors of interface 402. In this way, such measurements may be stored and. made available to other system components which may be communicatively coupled with interface 402.

System 400 further includes a controller that includes one or more processing devices and memory. Controller 404 may be configured to receive data from interface 402 (e.g., data from one or more EEG sensors associated with EEG data measured by the EEG sensors) and determine a current alpha power of the user. The current alpha power may be compared to a historical alpha power exhibited by the user after a night of sufficient sleep quality. Such a historical alpha power may be a reference reading (e.g., a measurement indicating adequate sleep, a personal best reading, or another such reading of the user). The current alpha power may be compared to the historical alpha power to determine whether the user has had adequate sleep. Accordingly, controller 404 is configured to receive data from sensors and determine whether the user has received adequate sleep. Furthermore, controller 404 may receive data from additional sensors to determine if the measurement is being properly taken.

Controller 404 may also be confirmed to implement learning estimator models that learn state changes and estimate them. Such learning estimator models may also learn changing system parameters, and estimate the improvement/retrograde of behavioral/functional responses. Such observers and estimators may be used to identify and/or infer state signatures and parameters associated with brain states. For example, examples of brain state signatures may include certain lower frequency oscillations mediated with or coupled to higher frequency oscillations (delta to alpha, alpha to gamma, theta to mu, alpha to high frequency band) that correspond with various levels of cognitive ability and various types of cognitive conditions to detect and identify signatures indicative of particular types of cognitive performance or cognitive conditions.

Various components of system 400 may further communicate with client device 408. Client device 408 may, for example, receive data from controller 404 through one or more wired and/or wireless communications interfaces (e.g., USB, WiFi, Bluetooth, and/or other communications protocols). Client device 408 may then accordingly provide notifications (e.g., adequate or inadequate sleep). Client device 408 may further receive commands from a user and provide such commands to controller 404 and provide instructions for controller 404 and/or interface 402. In various implementations, client device 408 may be any one of various computing devices such as laptop or desktop computers, smartphones, personal digital assistants, portable media players, tablet computers, or other appropriate computing devices that can be used to communicate over a global or local network, such as the Internet.

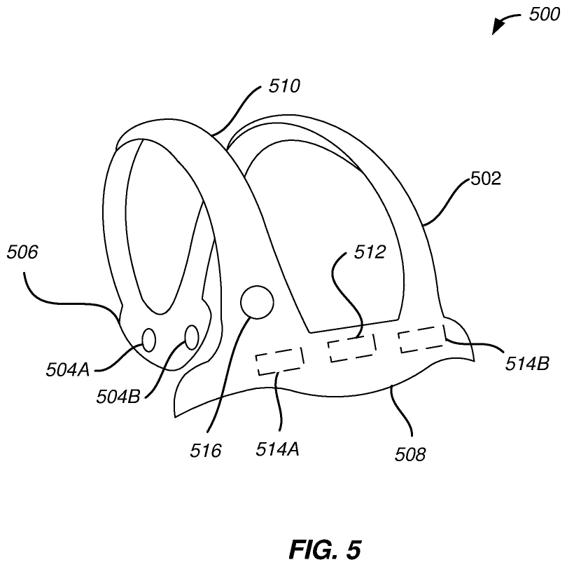

FIG. 5 illustrates an example of a sleep quality measuring device, in accordance with some embodiments. FIG. 5 illustrates a sleep quality measuring device 500 that includes a body 502, EEG sensors 504A and 504B, eye sensors 514A and 514B, controller 512, and interface 516.

Body 502 may be configured to couple to a head of a user, allowing for sleep quality measuring device 500 to be worn by the user. Body 502 may include body occipital portion 506, eye portion 508, and bridge 510 coupling together occipital portion 506 and eye portion 508.

In various embodiments, EEG sensors may be disposed within various portions of body 502. However, as described herein, EEG sensors for sensing alpha power, which is the most indicative and sensitive for determining whether a user has obtained adequate sleep, should be placed to at least contact the occipital aspect of a user's head. Thus, for example, EEG sensors 504A and 504B are located within body occipital portion 506 and disposed to contact an occipital aspect of the user's head, such as bilaterally contact each of O1 and O2. Various embodiments may include EEG sensors in other portions of body 502 (e.g., as described in FIG. 4B), but in the embodiments described herein, each sleep quality measuring device 500 includes at least one EEG sensor disposed to contact the occipital aspect of the user's head.

The EEG sensors may be coupled to controller 512 through, for example, one or more cables threaded or otherwise disposed within body 502. In various embodiments, the EEG sensors are passive electrodes distributed to measure the regions of interest (e.g., O1 or O2). Body 502 may be adjustable to ensure firm connections between the sensors (e.g., the electrodes) and the scalp of the user.

EEG sensors 504A and 504B are communicatively coupled to controller 512 through one or more wired and/or wireless data connections. Thus, for example, EEG sensors 504A and 504B may be configured to receive EEG power readings from the user and provide data associated with such readings to controller 512. Controller 512 may include micro-components configured to amplify the EEG signals, perform a fast Fourier transform (FTT) analysis on the amplified signals, determine whether the user has had adequate sleep, and provide an output (e.g., a visual or audible output) communicating whether the user has had adequate sleep.

In various embodiments, controller 512 may include memory configured to store reference data for comparison. Thus, for example, controller 512 may store reference data of a night when the user has been determined to have adequate sleep (e.g., from a previous test), store data directed to a personal best data (e.g., highest EEG reading) of the user, and/or store data related to other data (e.g., data correlated with the user or based on factors of the user such as age, height, weight, and other such factors) associated. with whether the user has obtained sufficient sleep. Controller 512 may compare the stored data to the detected data and, if the detected data is within a threshold percentage of the stored data (e.g., the detected EEG power is within 70%, 75%, 80%, 90%, 100%, 110%, or another percentage of the EEG power of the stored data), may determine that the user has had adequate sleep. Otherwise, controller 512 may determine that the user has had inadequate sleep. In various embodiments, a sleep adequacy rating may be determined. The sleep adequacy rating may be a simple adequate or inadequate sleep rating, may be a percentage output of the amount of EEG power determined, may be an inadequate, fair, adequate, great rating, or may be another such rating.

In certain embodiments, the output may be provided to a client device such as a smartphone, computer, or other device of the user, and the output may then be correspondingly displayed on the device. In other embodiments, sleep quality measuring device 500 may include an interface such as a screen or speaker that communicates the output to the user.

Eye portion 508 may be configured to be disposed over or proximate the eyes of a user. In various embodiments, eye portion 508 may include eye sensors 514A and 514B. Eye sensors 514A and 514B may be, for example, visual or thermal cameras that are positioned proximate to the user's eyes (e.g., proximate the brow or temple of the head of the user) when sleep quality measuring device 500 is worn by the user and configured to provide eye movement data to controller 512. Thus, eye sensors 514A and 514B may be configured to detect eye movement which would be used for noise and movement rejection (e.g., eye movement beyond a threshold may cause the detected EEG data to be rejected).

Interface 516 may be configured to receive user inputs and/or provide outputs to the user and may be communicatively coupled to controller 512. Thus, for example, interface 516 may be a button, a graphical display (e.g., a screen or other type of display), a speaker, a haptic feedback component (e.g., a piezoelectric component configured to vibrate sleep quality measuring device 500), or another such interface configured to receive and/or provide feedback to the user. In certain embodiments, sleep quality measuring device 500 may be configured to record a sample of eyes opened EEG followed by eyes closed EEG. In such embodiments, sleep quality measuring device 500 may be configured to identify eyes open EEG by having the user first press a button. Once the eyes opened EEG is measured, sleep quality measuring device 500 may then produce a sound or provide a vibration to be felt by the user. The user may then press the button again and close the user's eyes to have the sleep quality measuring device 500 measure eyes closed EEG. Eyes opened and eyes closed EEG may then both be measured.

In certain embodiments, sleep quality measuring device 500 may be configured to determine whether EEG sensors 504A and 504B arc recording EEG power or noise. For example, if the user is sitting next to a large electrical object, the electrical noise produced by the object may interfere with the EEG recording. Inadequate contact between the EEG sensors and the scalp may provide noisy recordings as well. Thus, for example, controller 512 may analyze the EEG data output from the EEG sensors 504A and 504B. If the data follows a general form associated with the user (e.g., amplitude, wavelength, and other characteristics), controller 512 may determine that the data is directed to EEG data. In certain such embodiments, the amplitude may he higher or lower depending on the EEG power of the user, but the general shape of the curve may be similar to that of stored historical data. However, if the shape of the data is substantially different (e.g., of a different shape of curve), controller 512 may determine that the readings arc noise or other inadequate. Sleep quality measuring device 500 may then output a message inadequate that the data measured is insufficient (e.g., output through an interface on sleep quality measuring device 500 or communicated to a client device).

Certain embodiments of sleep quality measuring device 500 may be configured to be worn by a user to sleep. In such embodiments, controller 512 may be configured to determine when a user has awoken and automatically measure EEG power in response to determining that the user has awoken. Thus, eye sensors 514A and 514B may be configured to determine when a user's eyes have opened for a threshold period of time, indicating that the user has awoken. If controller 512 has determined that the user has awoken, EEG power may then he measured if conditions are appropriate (e.g., if the user's eyes has closed to allow for measuring of eyes closed EEG power). In certain embodiments, sleep quality measuring device 500 may output an indication (e.g., a vibration generated by a piezoelectric device within sleep quality measuring device 500 or an audible sound) or communicate such an indication to a client device to indicate to the user that sleep quality measuring device 500 has determined that the user has awoken. Such an indication may communicate to the user that the user should go through the sleep quality measuring cycle (e.g., measuring of eyes closed and/or eyes open EEG power over a set period of time).

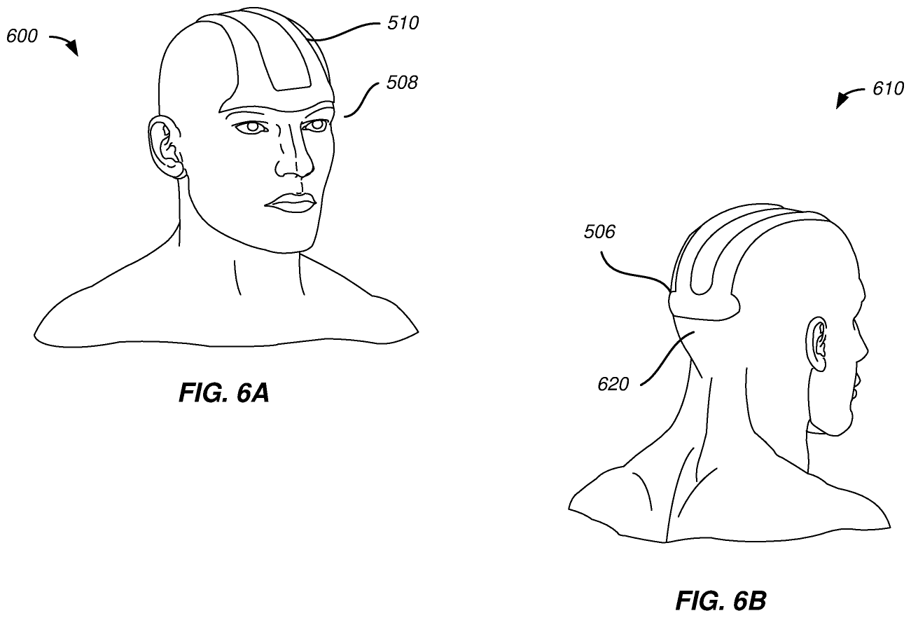

FIGS. 6A and 6B illustrates example of a user wearing a sleep quality measuring device, in accordance with some embodiments. FIGS. 6A and 6B illustrate the positioning of sleep quality measuring device 500 when worn on a head of a user. As shown in view 610 of FIG. 6B, occipital portion 506 and, thus, EEG sensors 504A and 504B are disposed proximate to occipital aspect 620 of the user's head. EEG sensors 504A and 504B include leads configured to contact the user's head. Furthermore, as shown in view 600 of FIG. 6A, bridge 510 is disposed over a parietal portion of the head of the user. Such a configuration may allow for sleep quality measuring device 500 to be worn when the user is sleeping, allowing for eye portion 508 to be disposed over the eyes of the user along the brow of the user and for eye sensors 514A and 514B to detect eye movements of the user. Other embodiments may include eye portion 508 to extend along the temples of the user to dispose eye sensors 514A and 514B along the temples of the user.

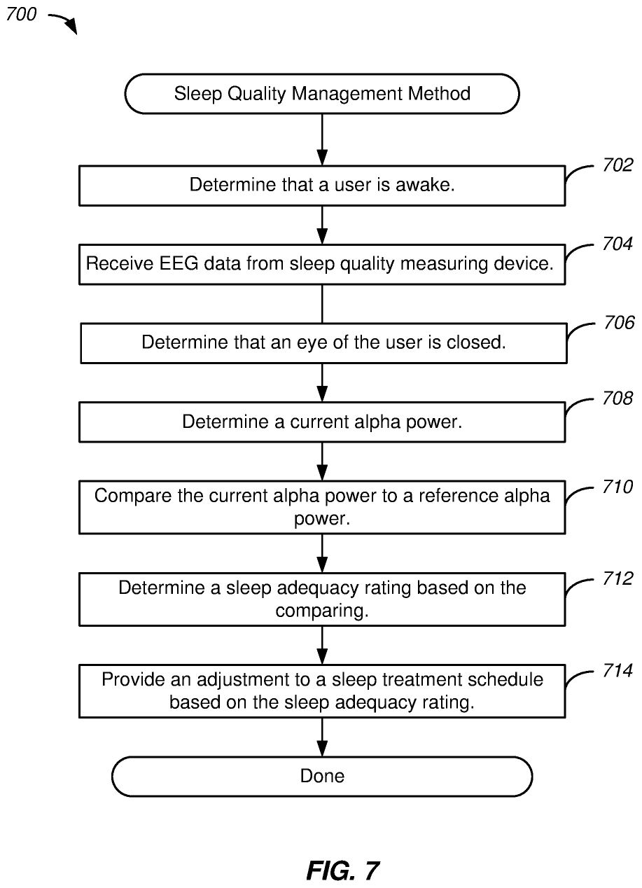

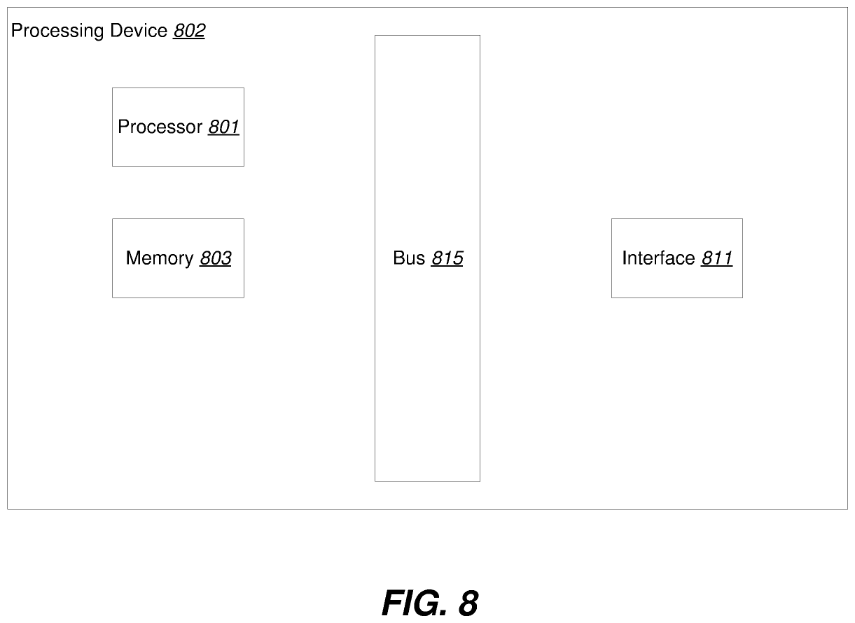

FIG. 7 is a flowchart illustrating a technique of using the sleep quality measurements, in accordance with some embodiments. FIG. 7 illustrates a sleep quality management method that may be used in conjunction with treatment methods to increase the quality of sleep experienced by a user.

In block 702, a determination may be made that the user is awake. Such a determination may be made by data from eye sensors as described herein, or through another technique. For example, the sleep quality measuring device may include one or more accelerometers and accelerometer data may indicate that the user has put on the sleep quality measuring device or has arisen out of bed, indicating that the user has awaken or a client device may communicate to the sleep quality measuring device that a wake up alarm has gone off. In another example, EEG sensors may determine that the user has put on the sleep quality measuring device and the EEG sensors are now contacting the user, indicating that the user has awakened.

In block 704, EEG data may be received from one or more EEG sensors of the sleep quality measuring device. The one or more EEG sensor may be contacting the head of the user at specified locations (e.g., an occipital aspect of the user's head, as described herein).

In certain embodiments, the EEG data must be measured within a threshold period of time (e.g., between 30 minutes to one hour after the user has awakened or within another such threshold) after a determination that the user has awakened. In various treatment or diagnosis techniques, measurement may be taken when the user is free of sleep associated drugs (e.g., sleeping pills or other such drugs) in order to receive an accurate reading.

In block 706, the sleep quality measuring device may determine that the eyes of the user are closed. For example, the user may be trained to operate the sleep quality measuring device. The user may press a button or touchscreen on the sleep quality measuring device to indicate that the user's eyes are closed and that the sleep quality measuring device may measure eyes closed EEG data of the user. In other embodiments, block 706 may be optional.

In various other embodiments, the sleep quality measuring device may measure both eyes opened and eyes closed EEG power. In such embodiments, the sleep quality measuring device may determine whether the user's eyes are opened or closed (e.g., from feedback from the user) and accordingly mark the EEG data measured at that time. Thus, the sleep quality measuring device may then detect both eyes open and eyes closed EEG power.

Based on the EEG data, a current alpha power of the user may be determined in block 708. The current alpha power may be determined from the EEG data as described herein. The current alpha power may be compared to a reference alpha power in block 710. The reference alpha power may be as described herein. In certain embodiments, both an eyes open and an eyes closed alpha power of the user may be determined in block 708 and both the eyes open and eyes closed power may be compared in block 710. In such embodiments, the user may indicate whether eyes are open or closed to the sleep quality measuring device (e.g., by the pressing of a button). Other embodiments may only determine and compare eyes closed alpha power

Based on the comparison, a sleep adequacy rating may he determined in block 712. Thus, whether the user has had adequate sleep may be determined. In certain embodiments, the sleep adequacy rating may be determined based on whether the current alpha power is within a percentage or has exceeded the reference alpha power. The sleep adequacy rating may then be output.

Based on the sleep adequacy rating, an adjustment to a sleep treatment schedule for the user may be determined in block 714. Thus, for example, certain users may be insomniacs. As insomniacs may find it difficult to determine whether they have obtained adequate sleep, the treatment for such insomniacs may be adjusted based on the determined sleep adequacy rating. Furthermore, sleep apnea may also be treated based on the determined sleep adequacy rating. Such adjustments may include, for example, a change in a drug schedule of the user, a change to a stimulus control therapy of the user, a change to a relaxation schedule of the user, a change to a sleep restriction of the user, and/or a change to a light therapy of the user.

In certain embodiments for treatment of sleep apnea, the sleep quality measuring device may determine an effectiveness of continuous positive airway pressure (CPAP) treatment. Thus, the sleep quality measuring device may communicate with a CPAP machine. When the CPAP machine is used for treatment, data may be communicated to the sleep quality measuring device indicating that the CPAP machine is in use. The sleep quality measuring device may then measure a sleep adequacy of the user after the user has awakened to determine the quality of treatment by the CPAP machine.

FIG. 8 illustrates a block diagram of a computer system capable of implementing various processes described herein, in accordance with sonic embodiments. FIG. 8 illustrates an example of a computer system or a processing device 802 that can be used with various embodiments. According to particular example embodiments, a processing device 802 suitable for implementing particular embodiments of the present invention includes a processor 801, a memory 803, an interface 811, and a bus 815 (e.g., a PCI bus). The interface 811 may include separate input and output interfaces, or may be a unified. interface supporting both operations. When acting under the control of appropriate software or firmware, the processor 801 is responsible for tasks such as brain stimulation described above. Various specially configured devices can also be used in place of a processor 801 or in addition to processor 801. The complete implementation can also be done in custom hardware. The interface 811 is typically configured to send and receive data packets or data segments over a network. Particular examples of interfaces the device supports include Ethernet interfaces, frame relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as packet switching, media control and management.

According to particular example embodiments, the processing device 802 uses memory 803 to store data and program instructions and maintain a local side cache. The program instructions may control the operation of an operating system and/or one or more applications, for example. The memory or memories may also be configured to store received metadata and batch requested metadata.

Because such information and program instructions may be employed to implement the systems/methods described herein, the present invention relates to tangible, machine readable media that include program instructions, state information, etc. for performing various operations described herein. Examples of machine-readable media include hard disks, floppy disks, magnetic tape, optical media such as CD-ROM disks and DVDs; magneto-optical media such as optical disks, and hardware devices that are specially configured to store and perform program instructions, such as read-only memory devices (ROM) and programmable read-only memory devices (PROMs). Examples of program instructions include both machine code, such as produced by a compiler, and files containing higher level code that may be executed by the computer using an interpreter.

While the present disclosure has been particularly shown and described with reference to specific embodiments thereof, it will be understood by those skilled in the art that changes in the form and details of the disclosed embodiments may be made without departing from the spirit or scope of the invention. Specifically, there are many alternative ways of implementing the processes, systems, and apparatuses described. It is therefore intended, that the invention be interpreted to include all variations and equivalents that fall within the true spirit and scope of the present invention. Moreover, although particular features have been described as part of each example, any combination of these features or additions of other features are intended to be included within the scope of this disclosure. Accordingly, the embodiments described herein are to be considered as illustrative and not restrictive.

Although many of the components and processes are described above in the singular for convenience, it will be appreciated by one of skill in the art that multiple components and repeated processes can also he used to practice the techniques of the present disclosure.

What is claimed is:

1. A sleep quality measuring device, comprising:
a body, configured to couple to a head of a user, wherein the body comprises a body occipital portion formed to be disposed next to an occipital aspect of the head of the user;
an electroencephalogram (EEG) sensor, disposed on the body occipital portion and configured to contact the occipital aspect when the sleep quality measuring device is worn by the user to detect EEG readings from the occipital aspect of the head of the user and output corresponding EEG data; and
a controller configured to perform operations comprising:
receiving the EEG data from the EEG sensor;
determining, from the EEG data, a current alpha power of the user;
comparing the current alpha power to a reference alpha power;
determining a sleep adequacy rating based on the comparing; and
outputting the sleep adequacy rating.

2. The sleep quality measuring device of claim 1, wherein the EEG sensor is a first EEG sensor and the sleep quality measuring device further comprises:
a second EEG sensor configured to contact the head of the user outside of the occipital aspect of the user.

3. The sleep quality measuring device of claim 1, further comprising:
an eye sensor configured to output eye movement data to the controller, wherein the operations further comprise:
receiving the eye movement data, wherein the determining the current alpha power of the user is further based on the eye movement data.

4. The sleep quality measuring device of claim 3, wherein the body further comprises an eye body configured to be disposed proximate a brow or temple of the user, and wherein the eye sensor is disposed on the eye body and configured to be disposed proximate to the brow or the temple of the user.

5. The sleep quality measuring device of claim 4, wherein the body further comprises a bridge coupling the eye body to the body occipital portion, wherein the bridge is configured to be disposed over a parietal portion of the head of the user when the sleep quality measuring device is worn by the user.

6. The sleep quality measuring device of claim 1, wherein the reference alpha power is determined when an eye of the user is closed.

7. The sleep quality measuring device of claim 6, wherein the operations further comprise:
receiving eyes open EEG data from the EEG sensor; and
determining, from the eyes open EEG data, an eyes open alpha power of the user.

8. The sleep quality measuring device of claim 7, wherein the operations further comprise:
comparing the eyes open alpha power to an eyes open reference alpha power, wherein the sleep adequacy rating is further based on the comparing the eyes open alpha power to the eyes open reference alpha power.

9. The sleep quality measuring device of claim 1, wherein the outputting the sleep adequacy rating is through an audible and/or visual output.

10. The sleep quality measuring device of claim 1, wherein the outputting the sleep adequacy rating comprises transmitting the sleep adequacy rating to an associated device.

11. The sleep quality measuring device of claim 1, wherein the EEG sensor comprises a plurality of EEG sensors, the plurality of EEG sensors configured to bilaterally contact the occipital aspect of the user when the sleep quality measuring device is worn by the user.

12. The sleep quality measuring device of claim 11, wherein all EEG sensors of the sleep quality measuring device are configured to contact the occipital aspect of the user.

13. The sleep quality measuring device of claim 1, wherein the EEG readings comprises alpha waves of between 8 to 12 Hertz.

14. The sleep quality measuring device of claim 1, wherein the controller is configured to communicate with a continuous positive airway pressure (CPAP) component, and wherein the operations further comprise:
determining an effectiveness of CPAP treatment based on the sleep adequacy rating.

15. A method comprising:
receiving, within a threshold period of time after a user has awoken when the user is free of sleep associated drugs, electroencephalogram (EEG) data from a sleep quality measuring device worn on a head of the user, wherein the sleep quality measuring device is formed to dispose an EEG sensor of the sleep quality measuring device to contact an occipital aspect of the head of the user when the sleep quality measuring device is worn on the head of the user;
determining, from the EEG data, a current alpha power of the user;
comparing the current alpha power to a reference alpha power;
determining a sleep adequacy rating based on the comparing; and
providing an adjustment to a sleep treatment schedule based on the sleep adequacy rating.

16. The method of claim 15, wherein the adjustment comprises a change in a drug schedule of the user.

17. The method of claim 15, wherein the adjustment comprises a change to a stimulus control therapy of the user, a relaxation schedule of the user, a sleep restriction of the user, and/or a light therapy of the user.

18. The method of claim 15, further comprising:
determining that an eye of the user is closed, wherein the determining the current alpha power is based on the determining that the eye of the user is closed.

19. The method of claim 15, further comprising:
determining that the user is awake, wherein the receiving the EEG data is based on the determining that the user is awake.

20. The method of claim 15, wherein the sleep treatment schedule is associated with evaluation of insomnia and/or sleep apnea.
